(19) United States
(12) Patent Application Publication
(10) Pub. No.: US 2018/0185063 A1
(43) Pub. Date: Jul. 5, 2018

(54) EXFOLIATING ABRASIVE SHOWER OR BATH MAT

(71) Applicant: Josephine Moretti DiMitri, Staten Island, NY (US)

(72) Inventor: Josephine Moretti DiMitri, Staten Island, NY (US)

(21) Appl. No.: 15/396,885

(22) Filed: Jan. 3, 2017

(51) Int. Cl. A61B 17/54; A47K 3/00

(52) U.S. Cl. CPC A61B 17/54; A61B 2017/320004; A47K 3/001

(57) ABSTRACT

A device and method for removing dead skin from a person's foot comprising: a bath or shower mat comprised of flexible plastic, rubber or other similar materials. The mat comprises an upper surface and bottom surface. The upper surface comprised of an abrasive surface. The mat is further comprised of a device for holding the bottom surface against a floor.

### FIELD OF THE INVENTION

The present invention relates to a foot exfoliating shower or bath mat.

### BACKGROUND OF THE INVENTION

U.S. Pat. No. 6,662,398 relates to a hands free foot scrubber. The bath mat includes a raised foot pad area having bristles on an upper surface and an enclosed refillable bladder containing liquid soap fitting within the under surface below the foot pad. Foot pressure causes soap to be released through tubular passages in the bladder engaging corresponding passages in the foot pad. Feet may be cleaned by scrubbing on the bristles without having to bend over or use the hands. The mat includes water drain holes and suction cups on the bottom of the mat and bladder which hold the components in place.

U.S. Pat. No. 5,575,034 relates to a foot cleaning and massaging mat system, comprising a mat in a generally rectangular configuration. The mat system is comprised of an upper planar surface and a lower planar surface which are coupled around their periphery with an outwardly directed projection. An aperture is formed in the center of the mat and extends completely therethrough. An upwardly extending recess is formed from the lower surface of the mat peripherally around the aperture. An insert is positioned within the recess and has a plurality of apertures in a symmetric configuration. A plurality of upstanding bristle-like members extend upwardly from the upper surface of the mat between the aperture and the periphery.

U.S. Pat. No. 6,253,407 relates to a foot scrubber designed to be placed on a shower floor. The foot scrubber consists of a base having an upper surface, a lower surface and side edges. From the upper surface extends upward a multiplicity of bristles. To use the scrubber, a person applies soap to the bristles and then rubs their foot on the bristles.

U.S. Pat. No. 6,530,096 relates to a foot rejuvenation shower apparatus. The device is constructed of a lower surface that is releasably engaged with a floor and an upper portion with a selectively applicable upper surface for removing dead skin of a foot of a user.

US patent publication no. 2006/0185071 relates to a therapeutic floor mat system and method. The system includes a mat having an aromatherapy cavity. The cavity may be formed to at least partially hold an aromatic substance. The mat may work as a shower mat.

U.S. Pat. No. 8,505,551 relates to an exfoliating shower mat that claims a foamy cushioned material having a device for holding the mat to the surface of the floor and having a piece cut to fit over a drain or perforations to let the water and hair exit the mat.

Pedi-Quick Step-N-Smooth shower foot file relates to a device which allows a user to file their feet while standing in the shower. The device quickly reduces corns, calluses and rough, dry skin from bottoms of feet. The device has a non-skid backing.

The device is placed on the shower floor. The water is turned on to wet the file. A user steps on the device with two feet. Using one foot at a time, a user moves one foot toward themselves to file skin, making sure the repeated strokes are in the same direction. Change feet and repeat the process. The product is rinsed after use, and the mat is stood upright against a shower wall to allow it to air dry.

U.S. Pat. No. 8,505,551 relates to a device for removing dead skin from a person's foot comprising a single layer not made of foamy material having an upper surface comprising a rough flat surface, bullnose edging on one or both ends of mat, and a device for holding the mat to the floor.

### SUMMARY OF THE INVENTION

The present invention relates to a mat that is used in the shower or bath to remove dead skin from a person's foot. The mat is comprised of a flexible plastic, rubber or other similar materials, the mat comprising an upper surface and bottom surface; the upper surface comprised of an abrasive surface; the abrasive surface being within a smooth surfaced edge that surrounds the mat. The mat is comprised of a device for holding the bottom surface against a floor.

It is an object of the present invention for the mat to be made of a foamy, cushioned, light weight material with an abrasive material on the surface. The mat further comprises an edging that surrounds the abrasive material. The dead skin is removed while a user is showering, they rub their feet on the abrasive surface without bending or use of the hands. The mat has an embossed pattern on its surface.

It is an object of the present invention for the material of the mat to be flexible. It is an object of the present invention for the mat to be comprised of rubber. It is an object of the present invention for the abrasive material to be comprised of aluminum oxide cloth. It is an object of the present invention for the mat to comprise two separate material: a top aluminum oxide cloth attached to rubber with suction cups on the bottom. It is an object of the present invention for the mat to have no beveled edges. It is an object of the present invention for the mat to have one or more beveled edges.

It is an object of the present invention for the mat to comprise, PVC, plastic, rubber, composite, soft-foam, silicone, or vinyl. It is an object of the present of the mat to be made of a recycled material. It is an object of the present invention for the mat to be made of a biodegradable material.

It is an object of the present invention for the abrasive material to be made of a synthetic material. It is an object of the present invention for the abrasive material to be made of a natural material. It is an object of the present invention for the abrasive material to comprise sand, rock, etc. It is an object of the abrasive material to be made of a recycled abrasive material.

It is an object of the present invention for the mat to be made by injection molding. It is an object of the present invention for the injection molding to comprise pre-heating the rubber before being injected into the mold. Since it is pre-heated, the rubber flows more readily into the mold, allowing for higher cure temperatures, and shortening the curing cycle.

It is an object of the present invention for the mat to be injected with air.

It is an object of the present invention for the mat to include suction cups or a suction channel on the bottom of the mat which holds the mat in place. It is an object of the present invention for the mat to have antibacterial and mildew resistant properties. It is an object of the present invention for the mat to be produced out of antimicrobial materials.

It is an object of the present invention for the mat to be rolled up when not in use. It is an object of the present invention for the mat to be hang dried. It is an object of the present invention for the mat to be big enough so that a person can stand on the mat with both feet.

The mat is not placed over a drain so that there are no pieces cut out of the mat. The mat is a continuous piece of material without any perforations or pieces cut out.

It is an object of the present invention for the mat to be made of two or more pieces. One bottom part of the mat to be made of a material with an adhesive device so that it stays in the bath or shower. A top layer is added to the bottom layer. The top layer can consist of one or two layers. The top layer consists of an abrasive material, which can be added directly to the bottom layer or attached to a layer which is then attached to the bottom layer which attached to the bath or shower. An adhesive material is placed between the bottom part of the mat and the layer or layers containing the abrasive layer so that they stay together. In this manner when the abrasive layer is no longer abrasive, it can be replaced with a new layer. It is an object of the present invention for the top abrasive layer to have a sticky backing to be placed on the bottom rubber layer.

It is an object of the present invention for the mat to comprise one piece with a coating application for the abrasive material to be on top of the mat. It is an object of the present invention for the mat to have an embossed pattern, e.g. ridges.

It is an object of the present invention to bond the top layer abrasive cloth to the bottom layer of the mat including the suction cups. It is an object of the present invention for the boding to comprise a heat process. It is an object of the present invention for the boding to comprise a pressure process. It is an object of the present invention for the bonding to include both heat and pressure. It is an object of the present invention for the bonding to be done by an adhesive additive.

It is an object of the present invention to produce the mat via compression molding.

It is an object of the present invention for the compression molding to comprise pressure and heat to mold the rubber.

It is an object of the present invention for the mat to be produced via transfer molding. Transfer molding is similar to compression molding but has a transfer system on the top of the mold that feeds the rubber into multiple mold cavities when the press is closed.

It is an object of the present invention for the compression mold to require one or two cavities depending on the requirement of addling the abrasive material on the top (surface) of the mat.

It is an object of the present invention for the mat to have an embossed texture on the top surface of the finished mat. The embossing may vary in design. It is an object of the present invention for the abrasive material to be a coated aluminum oxide cloth backing, or optional bonding of abrasive material mineral or synthetic.

It is an object of the present invention for an abrasive surface material to be applied via a spray coating. It is an object of the present invention for the abrasive surface material to be adhered or bonded to the mat.

It is an object of the present invention to produce the mat via injection molding.

It is an object of the present invention for the injection mold to have an embossed texture incorporated in the mold.

It is an object of the present invention for the abrasive to have different gradients on the surface of the mat, so that a user can use a lighter abrasive or a heavier abrasive depending on where they are standing on the mat.

It is an object of the present invention for the mat to have a beveled edge at one or more edges of the mat. It is an object of the present invention for the mat to have one or more bull nose features at one or more edges of the mat.

It is an object of the present invention for the mat to have a center portion with the abrasive material so a user can scrape the dead skin off their feet by moving their feet in a back and forth manner over the abrasive material.

It is an object of the present invention for the system to include a mesh bag. It is an object of the present invention for the bag to be made of nylon. It is an object of the present invention for the bag to have antibacterial and mildew resistant properties. It is an object of the present invention for the mat to be able to be rolled up and placed in the bag and drip dried after use.

It is an object of the present invention for the mat to be in the shape of a square with rounded corners so that it fits in the bath or shower and has enough surface area to properly scrape the bottom of a user's feet. It is an object of the present invention for the mat to be approximately 12″ to 16″ in size.

It is an object of the present invention for the mat to have a non-slip surface.

It is an object of the present invention for the mat to have smooth surfaces on the edges and the abrasive material in the center of the mat.

It is an object of the present invention for the mat to be made of a non-latex material.

The present invention relates to a method for scraping dead skin off of a person feet, both bottom and sides. First a user can place the mat having the suction cups on the floor of a bath or shower. The person then rubs their feet against the abrasive part of the mat removing dead skin from the bottom of their feet. Then a user can place the mat against the side of the tub or shower wherein the mat stays attached against the side or wall, and rubs the side of their feet against the abrasive part of the mat removing dead skin from the side of their feet.

It is an object of the present invention for the abrasive material to comprise: aluminum oxide, silicon carbide, zirconia alumina, ceramic abrasives, or a combination of same.

### BRIEF DESCRIPTION OF THE DRAWINGS

FIG. 1 is a top view of the device of the present invention.

FIG. 2 is a bottom view of the device of the present invention.

FIG. 3 is a side view of the bag for holding the device of the present invention.

FIG. 4 is a top view of tooling for constructing an embodiment of the device of the present invention.

### DETAILED DESCRIPTION OF THE INVENTION

The present invention relates to a bath mat in the shower or bath tub to remove dead skin from a person's foot. The mat is made of a foamy, cushioned, light weight material with an abrasive surface. Dead skin is removed while a user is showering. A user rubs their feet on the abrasive surface without bending or use of the hands. This causes the dead skin to be removed from a person's feet.

The mat includes an adhesive or holding part for holding the device to the shower or bathtub floor. In a preferred embodiment, the mat includes suction cups or a suction channel on the bottom of the mat which holds the mat in place. In a preferred embodiment, the mat has antibacterial and mildew resistant properties.

In an embodiment, the mat is rolled up after use. In an embodiment the mat is hang dried. In a preferred embodiment, the mat is big enough so that a person can stand on the mat with both feet.

In an embodiment, the mat is made of two pieces. One bottom part of the mat is made of a light weight foam material with an adhesive device so that it stays in the bath or shower. A top layer is added to the bottom layer. The top layer consists of a sandy or abrasive material. An adhesive material is placed between the two layers so that they stay together. In this manner when the abrasive layer is no longer abrasive, it can be replaced with a new layer.

In an embodiment, the mat has a beveled edge.

The present invention further comprises a mesh bag. In an embodiment, the bag is made of nylon. In an embodiment, the bag has antibacterial and mildew resistant properties. In an embodiment, the mat is rolled up and placed in the bag and drip dried after use.

In an embodiment, the mat is in the shape of a square with rounded edges. In an embodiment, the mat is approximately 12″ to 16″ in size.

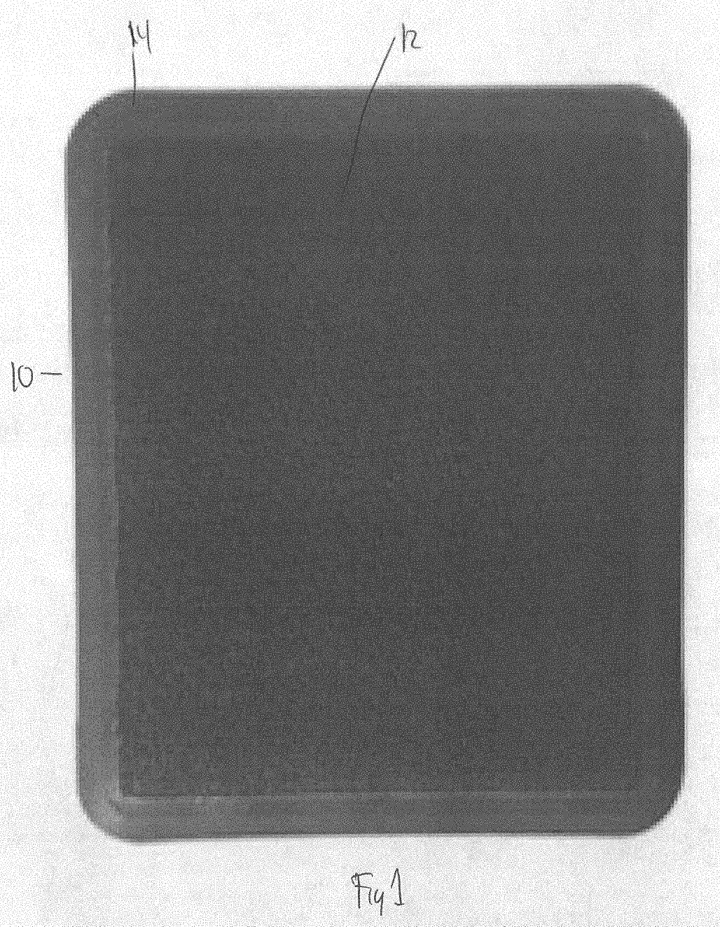

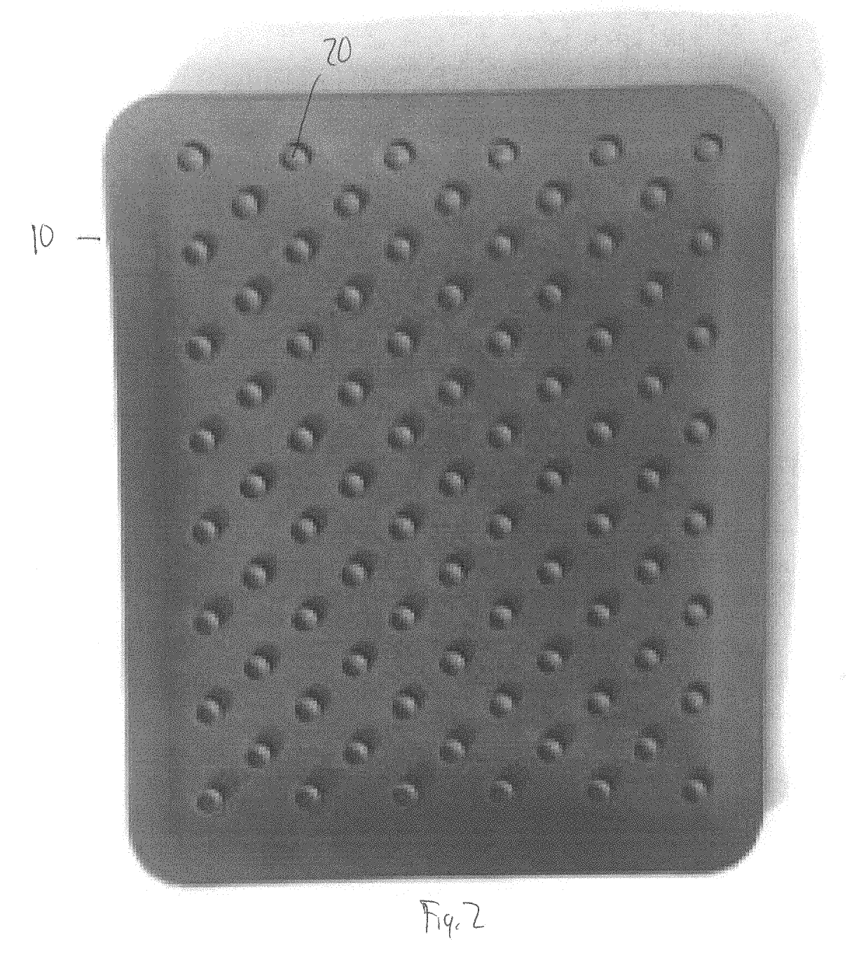

FIG. 1 shows the mat 10 from a top view. The mat 10 has an abrasive surface 12. The abrasive surface 12 is within a smooth outer edge 14 that surrounds the abrasive surface 12. FIG. 2 is a bottom view of the mat 10. The device 10 has suction cups 20 beneath the abrasive surface 12 for holding the mat 10 to the bath or shower floor or sides.

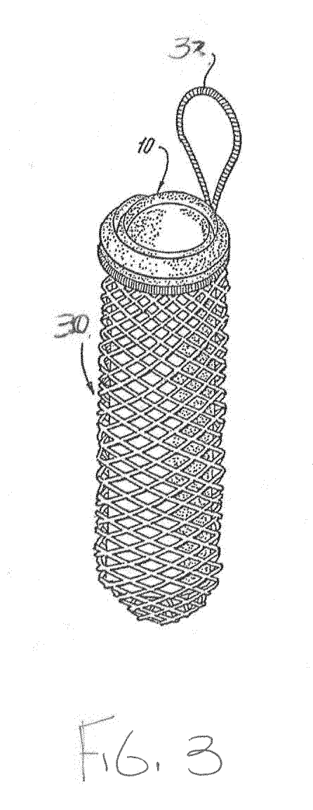

FIG. 3 shows the mesh bag 30 for holding the device 10. The bag 30 has a loop string 32 that is pulled closed and then can be hung on a hook or any door handle.

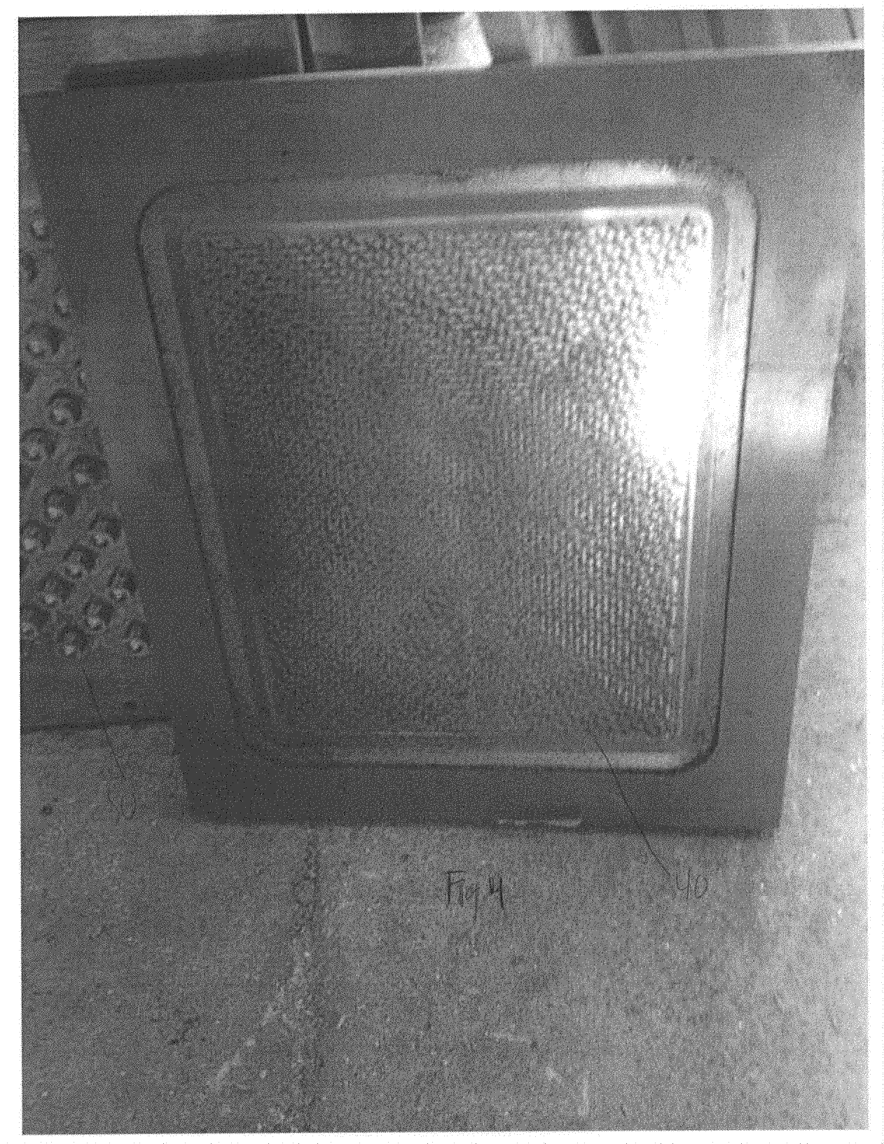

FIG. 4 shows tooling 40 for making the top of the mat and tooling 50 for making the bottom of the mat.

1. A bath or shower mat for removing dead skin from a person's foot comprising:
a mat comprised of a flexible plastic, rubber or other similar materials;
said mat comprising an upper surface and bottom surface;
said upper surface comprised of an abrasive surface;
said abrasive surface being within a smooth surfaced edge that surrounds said mat;
said mat comprised of a device for holding said bottom surface against a floor.

2. The mat of claim 1 wherein said device for holding said bottom surface against a floor comprises suction cups or a suction channel.

3. The mat of claim 1 wherein said mat comprises antibacterial and mildew resistant properties.

4. The mat of claim 1 wherein said mat is rolled up after use.

5. The mat of claim 1 wherein said mat is hang dried after use.

6. The mat of claim 1 wherein said mat comprises PVC, plastic, rubber, composite, soft-foam, silicone, or vinyl.

7. The mat of claim 1 wherein said abrasive material is comprised of aluminum oxide cloth, silicon carbide, zirconia alumina or ceramic abrasives.

8. The mat of claim 1 wherein said upper surface is comprised of an embossed pattern.

9. A bath or shower mat for removing dead skin from a person's foot comprising:
said mat comprised of two sections, a top section and a bottom section;
said bottom section comprised of a flexible plastic, rubber or other similar material with an adhesive or holding part so that said mat stays against a floor of a bath or shower;
a top section added to said bottom section;
said top section comprised of an abrasive material;
said top section comprised of one or more layers;
an adhesive material placed between said top section and said bottom section for holding said two sections together.

10. The mat of claim 4 wherein said mat is placed in a mesh bag after it is rolled up.

11. The mat of claim 10 wherein said bag is pulled closed and hung on a hook or any door handle.

12. A bath or shower mat for removing dead skin from a person's foot comprising:
said mat comprised of two separate materials;
a top material comprised of an aluminum oxide, silicon carbide, zirconia alumina or ceramic abrasives;
a bottom material comprised of rubber having suction cups at bottom side of said bottom material;
said top material attached to said bottom material.

13. The mat of claim 12 wherein said rubber is comprised of recycled material.

14. The mat of claim 12 wherein said mat is biodegradable.

15. The mat of claim 12 wherein said top material is bonded to said bottom material by a heat process, pressure process, or combination of said heat process and said pressure process.

16. The mat of claim 12 wherein said mat is made via a compression mold.

17. The mat of claim 12 wherein said mat is made via injection molding.

18. The mat of claim 12 wherein said top material comprises different gradients of abrasiveness.

19. A method for removing dead skin off of a person's bottom and sides of feet comprising:
placing a mat having suction cups on bottom of said mat onto floor of a bath or shower, said top of said mat comprising an abrasive surface;
rubbing bottom of said feet against said abrasive surface;
removing dead skin from bottom of said feet;
removing mat from said floor and placing said mat onto side of said bath or shower;
rubbing side of feet against said abrasive surface;
removing dead skin from side of said feet.

20. The mat of claim 1 wherein said mat is comprised of a foamy, cushioned and light weight material.
